(19) United States
(12) Patent Application Publication
(10) Pub. No.: US 2024/0376524 A1
(43) Pub. Date: Nov. 14, 2024

(54) INTEGRATED NUCLEIC ACID ANALYSIS SYSTEM AND METHOD FOR MEASURING TARGET NUCLEIC ACID IN SAMPLE

(71) Applicants: Shanghai Hengxin BioTechnology Limited, Shanghai (CN); Ningbo Hengxin BioTechnology Limited, Ningbo (CN)

(72) Inventors: Chuanyong WU, Shanghai (CN); Li ZHOU, Shanghai (CN); Zhifang WANG, Shanghai (CN)

(73) Assignees: Shanghai Hengxin BioTechnology Limited, Shanghai (CN); Ningbo Hengxin BioTechnology Limited, Ningbo (CN)

(21) Appl. No.: 18/692,826

(22) PCT Filed: Nov. 1, 2021
(86) PCT No.: PCT/CN2021/127976

§ 371 Date: Mar. 17, 2024

(30) Foreign Application Priority Data
Sep. 17, 2021 (CN) 2021110945981

(51) Int. Cl. C12Q 1/6806; C12N 15/10; C12Q 1/6844

(52) U.S. Cl. CPC C12Q 1/6806; C12N 15/1013; C12Q 1/6844

(57) ABSTRACT

The present invention provides a method for integrating a nucleic acid analysis system and measuring at least one target nucleic acid in a sample. The integrated nucleic acid analysis system includes one DMF device, which includes a substrate, a cover plate, and an instrument for performing a series of operations including magnetic beads based nucleic acid extraction, purification, thermal amplification, and optical measurement of nucleic acids in samples in a DMF device. The nucleic acid extraction and purification are based on the magnetic bead method, which uses instruments to move magnetic beads in different functional areas of the digital microfluidic chip, such as lysis reservoirs, cleaning reservoirs, and elution reservoirs, etc. The present invention integrates sample processing and detection on the same microfluidic chip, and the entire process is automatically completed by the corresponding instrument. Its advantages include reducing manual errors, reducing cross contamination, and improving detection speed.

### TECHNICAL FIELD

The present invention relates to the field of biological diagnostics technology, specifically to a method for integrating a nucleic acid analysis system and measuring at least one target nucleic acid in a sample. The present invention utilizes digital microfluidics (DMF) devices to achieve the integration of magnetic bead extraction, purification, amplification, and measurement of nucleic acids.

### BACKGROUND OF THE INVENTION

Before conducting detection and analysis of a biological sample, a series of mechanical, thermal, electrical, magnetic, optical, or chemical processing steps are usually required. In modern life sciences and medical diagnostic applications, magnetic beads are often used for sample processing, such as extraction, separation, and purification of analytes. During the extraction process, the target analyte is chemically bound to the surface of the magnetic beads, and separation or purification can be achieved by using a pipette or allowing the liquid to flow through the magnetic beads fixed by a magnet to remove unwanted particles or liquids. By using a magnet, magnetic beads can also be focused and removed from their original liquid environment to remove unwanted particles or liquids. Magnetic beads are often used as carriers for nucleic acids, antigens, antibodies, catalysts, and proteins, and are widely used in DNA isolation, mRNA purification, protein purification, cell separation, immunoassay, biomolecular capture, etc.

There are many instruments or devices used for biochemical processing and analysis. When using these instruments or devices, a substantial portion of the samples or reagents required for biochemical reactions do not participate in the reaction or measurement, thus being wasted. In digital microfluidic (DMF) systems, the dead volume (the volume of liquid added to the system but not involved in reactions or detection) can be significantly reduced, sometimes even to zero. This means that the amounts of samples or reagents required for the experiment is the amount measured. This not only greatly reduces the cost of using samples and reagents, but also greatly shortens the time required for measurement and analysis, as smaller reaction volumes can shorten the mixing time between reagents and samples.

Many biochemical analysis systems currently require a large number of manual processing steps. Compared to this, systems based on digital microfluidics (instruments, devices, and methods, etc.) can provide a high degree of integration and automation, which can greatly reduce possible human errors and greatly improve diagnostics reliability and data quality.

In droplet based digital microfluidic systems, liquids are manipulated in a discrete format (droplets) in a two-dimensional space, and droplets can be individually manipulated, which is why they are called digital microfluidics. In DMF devices, the operating path of droplets can be defined during operation and can be dynamically changed. Compared to conventional pressure driven (through external pumps) or electric driven (through high voltage) channel-based microfluidics, DMF devices only require low electric voltages to control the droplets. The driving force in digital microfluidics is based on electrostatic effects, such as electrowetting or dielectrophoresis, which are usually not directly related to the specific biological samples being tested. This makes the design of digital microfluidics systems and the target analytes independent, hence, making them more versatile.

In recent years, digital microfluidic technology has attracted widespread attention due to its ability to handle individual droplets, as well as its advantages such as easy miniaturization, integration, and automation. Digital microfluidic technology reduces the use of reagents, simplifies experimental steps, and shortens measurement time, making it highly advantageous.

Polymerase Chain Reaction (PCR) fundamentally changes the scientific field. As a mature method, PCR requires a cycle of repeated heating and cooling of the reaction system, which includes specific DNA primers, dNTP (deoxyribonucleoside triphosphate), and thermally stable DNA polymerase (such as Taq DNA polymerase), Each temperature cycle theoretically doubles the number of potential target DNA molecules, leading to exponential amplification of the target sequence. This technology can amplify trace amounts of DNA or RNA in the sample to levels that can be measured and analyzed. PCR technology has been applied in many different fields, including virus load testing, foodborne pathogen quantification, clinical diagnostics, drug resistance analysis, and forensic science, etc. Using PCR technology, doctors and researchers can determine the source of viral infection by analyzing a single cell. Nowadays, PCR can be used to detect many infectious organisms, such as COVID-19, HIV-1, hepatitis B, hepatitis C, SARS virus, West Nile virus, Mycobacterium tuberculosis, etc.

In addition to PCR, nucleic acid amplification can also be achieved isothermally. There are many isothermal amplification methods that can perform DNA or RNA amplification at a specified temperature, such as SDA (Strand Displacement Amplification), NASBA (Nucleic Acid Sequence Based Amplification), TMA (Transcription Mediated Amplification), RCA (Rolling Loop Amplification), RPA (Recombinase Polymerase Amplification), LAMP (Loop Mediated Isothermal Amplification), and HDA (Helicase Dependent Amplification).

CRISPR (Clustered Regularly Interspaced Short Palindromic Repeats) is a series of DNA sequences found in prokaryotic genomes such as bacteria and archaea. These sequences come from DNA fragments of viruses that previously infected these prokaryotes and are used by these prokaryotes to detect and destroy similar viruses in subsequent infections.

Cas (CRISPR associated system) is a type of endonuclease. Cas proteins and gRNAs (guide RNA) can recognize and cleave specific nucleic acid molecules that complement this gRNA sequence. Some Cas proteins further cleave other nucleic acid molecules around them, including reporter molecules, which is the basis of CRISPR detection. The diagnostic technology based on CRISPR has become a new but very promising method due to its rapid nucleic acid detection ability and single base specificity.

In medical diagnosis, systems that can achieve sample input and result output have many advantages, such as being used for on-site diagnostics, less operator training, ensuring operator safety, and reducing cross contamination. In the field of nucleic acid diagnostics, although such systems exist, they generally have problems such as long detection time, low sensitivity, and few detection targets. Therefore, the present invention proposes an improvement scheme. In the present invention, we propose an integrated nucleic acid analysis system with high sensitivity, multiple detection targets, and short measurement time, as well as a method for measuring at least one target nucleic acid in a sample.

### SUMMARY OF THE INVENTION

On the one hand, the present invention provides an integrated nucleic acid analysis system capable of performing magnetic bead based nucleic acid extraction and purification, thermal amplification (such as isothermal amplification and PCR), and fluorescence measurement. The integrated nucleic acid analysis system comprises at least one DMF device for measuring at least one sample. The DMF device comprises a substrate with a first surface, on which electrowetting electrodes for droplet or liquid control is provided, and a top cover plate, wherein the top cover plate comprises a second surface parallel to the first surface and coordinated with the first surface to form a gap for liquid operation. The gap comprises a reservoir for nucleic acid extraction, an optional reservoirs (one or more) for nucleic acid cleaning, and a reservoir for nucleic acid elution, and the top cover plate is provided with ports (holes) for loading or unloading samples or reagents. An instrument is used to perform a series of operations on samples in the DMF device, including magnetic bead based nucleic acid extraction, purification, thermal amplification, and optical measurement of the target nucleic acids. The bottom plate includes a substrate with multiple electrowetting electrodes for controlling droplets or liquids, including dispensing droplets from the elution reservoir, and at least a portion of the bottom plate is optically transparent (including possible electrodes and dielectric layers at that location) for optical excitation and measurement. The top cover plate includes a surface that is basically parallel to the bottom plate, an inlet and outlet (ports or holes) for loading and unloading samples and reagents, and reservoirs for reagents storage or reactions. At least a portion of the surface of the top plate is conductive; On the other hand, some electrodes on the substrate form multiple paths for the purposes such as nucleic acid amplification. As an example, some electrowetting electrode regions on the bottom substrate contain freeze-drying reagents, including but not limited to polymerase (especially heat stable enzymes such as Taq polymerase and its variants), deoxyribonucleotide triphosphate (dNTP; usually a mixture of dCTP, dTTP, dGTP, and dATP), PCR primers, labeled probes, reverse transcriptase (if the target nucleic acid is RNA), exonucleases, Cas proteases, one or more guide RNAs that bind to specific target molecules.

A DMF devices typically include a device caddy, providing certain protection for the bonded bottom and top cover plates, and providing alignment when the device is being loaded to an instrument, and so ono. For example, in one example, the device caddy includes a liquid reagent cartridge, which includes: a) multiple liquid capsules containing sample processing or detection reagents and filler fluids, b) a fluid channel connecting the capsules to the liquid adding hole on the top cover plate, and c) a liquid inlet.

As an example, the gap of the DMF device has different heights at different locations, and when the gap height of the DMF device changes from one value to another, the corresponding change of the slope in the second surface slope is continuous. This helps the droplet operations, especially droplet dispensing or splitting.

As an example, the light transmittance of the transparent bottom plate of the DMF device is greater than 30% in the visible light range, such as greater than 50%, 70%, and even greater than 90%.

As an example, the first reservoir contains sample lysis solution and magnetic bead solution, the second reservoir contains magnetic bead washing solution, and the third reservoir contains nucleic acid elution solution.

As an example, the gap of the DMF device is filled with a filler liquid, such as mineral oil, silicone oil, paraffin liquid, fluorosilicone oil, etc.

As an example, the instrument comprises:
a) At least one mechanical module for loading and unloading DMF devices, such as a loading and unloading tray;
b) At least one voltage control module connected to the DMF device for providing electrical signals to the DMF device for operating liquids or droplets in the DMF device;
c) At least one magnet control module, located above or below the DMF device, for controlling one or more magnets, each magnet having at least two degrees of freedom of motion, that is, can move in two directions;
d) At least one temperature control module, used to control a specified region of the DMF device to the specified temperature;
e) At least one light source for optical excitation for nucleic acid detection in the DMF device;
f) At least one optical detection module for fluorescence measurement, used for optical measurement of at least one location on the DMF device.

As an example, the integrated nucleic acid analysis system further includes a touch screen display, which provides a graphical interface for users to perform experiments and display experimental status, and/or a central processing unit for integrated operation and control of the aforementioned modules.

As an example, the magnet is a focusing magnet.

The present invention also provides a method for measuring at least one target nucleic acid in a sample, comprising adding a sample to the DMF device of an integrated nucleic acid analysis system as described in any of the above schemes, and then performing a detection operation on the sample. The detection operation comprises the following steps: 1) Mix the sample with a lysis solution to lyse cells (or viruses, or tissues) in the sample to release the nucleic acids into the solution; 2) Add magnetic bead solution to the sample; 3) Mix the magnetic beads with the sample to capture the nucleic acids onto the surface of the magnetic beads; 4) Clean the magnetic beads in the cleaning buffer; 5) Elute the nucleic acids from the magnetic beads into the elution buffer to obtain the target nucleic acids; 6) Add amplification reagents to the target nucleic acids obtained after elution to perform thermal amplification of the nucleic acids; 7) Measure the fluorescence intensity from the target nucleic acids.

In an example, the steps to clean the magnetic beads are as follows: a) Using the magnet in the instrument, move the magnetic beads from the lysis reservoir, through the filler liquid, to the bead cleaning reservoir on the DMF device, and move the magnetic beads along a specified trajectory within the range of the bead cleaning reservoir; c) Move the magnetic beads through the filler liquid to the elution reservoir.

In further examples, steps a) and c) further include step b) moving the magnetic beads from the bead cleaning reservoir, through the filler liquid, to another bead cleaning reservoir, and moving the magnetic beads along a specified trajectory within this reservoir.

In an example, the steps to elute the nucleic acids from the magnetic beads are as follows: a) Move the magnet to a designated position at a certain distance from the DMF device, so that the magnetic beads are dispersed in the eluent; b) Move the magnet in the direction parallel to the DMF device in the specified trajectory within the range of the third liquid reservoir to assist in eluting nucleic acids from the magnetic beads; c) Move the magnet to the third reservoir close to the DMF device; d) Move the magnet along the specified trajectory at the third reservoir to gather the magnetic beads inside; e) Move the magnet close to the DMF device to the specified magnetic bead disposal location; f) Move the magnet away from the position of the DMF device.

The magnetic beads used for biological nucleic acids extraction are usually superparamagnetic microspheres with small diameters (ranging from nanometers to micrometers), which can quickly group together in a magnetic field. After the magnetic field is removed, they can be uniformly dispersed in the liquid and are not easy to settle (sink down). By encapsulating magnetic beads with elements such as silane groups, amino groups, or hydroxyl groups, etc., nucleic acids extraction can be achieved. Under the action of lysate, nucleic acids (DNA or RNA) in cells (or viruses, or tissues) are released, and surface modified magnetic beads specifically bind with nucleic acids to form magnetic bead nucleic acid complexes. Nucleic acid magnetic bead complexes often have non-specific adsorbed impurities that may affect the next step of detection and therefore need to be removed. The usual removal method is to use washing solution. The present invention provides a method of removing impurities from magnetic bead nucleic acid complexes by using a magnet to move magnetic beads along a specified trajectory in immiscible solutions.

As an example, thermal amplification and fluorescence intensity measurement from the target nucleic acids are carried out as follows: a) One or more droplets containing the nucleic acids are dispensed from the elution reservoir using the electrowetting electrodes; b) Move droplets along the specified path along the electrowetting electrode for nucleic acids amplification and optical measurement. All steps of the detection operation are preferably automatically completed by the instrument.

In a specific example, the thermal amplification is PCR amplification or isothermal amplification. In further examples, the PCR amplification is carried out by placing reaction droplets between different temperature regions on the DMF device and moving them along a specified trajectory, with the temperatures in the specified region remaining constant during the PCR reaction.

As an example, optical measurements are done after mixing a portion of the droplet complex with different reagents.

Compared to existing technologies, the integrated nucleic acid analysis system provided by the present invention has advantages such as high sensitivity, multiple targets detection, and short detection time, which can reduce manual errors, reduce cross contamination, and improve analysis speed.

To make the technical solution and advantages of the present invention more prominent, further explanation will be provided in conjunction with the accompanying drawings.

### BRIEF DESCRIPTION OF DRAWINGS

FIGS. 1A-1E show an exemplary regional side view of a DMF device containing liquid and magnetic beads, as well as the process of manipulating magnetic beads using a magnet.

FIG. 2A shows an exemplary top view of a DMF device with a reservoir, sample, and reagent loading ports.

FIG. 2B shows a physical top view of the DMF device in FIG. 2A.

FIG. 3 shows an exemplary layout diagram of the electrowetting electrodes on the substrate of the DMF device shown in FIG. 2A.

FIG. 4 shows a combined view of FIG. 2A and FIG. 3, and provides a schematic diagram of thermal amplification and optical measurement.

FIG. 5A-5C shows a schematic diagram of the different designs of the top cover plate for the gap height variation of the DMF device.

FIGS. 6A-6D show exemplary schematic diagrams of different functional modules of the integrated nucleic acid analysis system provided by the present invention, wherein FIG. 6A shows a temperature control module; FIG. 6B shows a magnet control module that can simultaneously control two magnets;

FIG. 6C shows the optical measurement module; FIG. 6D is a top view of four temperature control modules and two magnets (part of the magnet control module).

FIGS. 7A and 7B show exemplary structural diagrams of the integrated nucleic acid analysis system provided by the present invention.

FIG. 8 shows an exemplary flowchart of magnetic bead based nucleic acid extraction and quantitative PCR detection implemented using the integrated nucleic acid analysis system provided by the present invention.

FIG. 9 shows a top view of the integrated nucleic acid analysis system provided for the present invention in another example.

FIG. 10 shows an exemplary flowchart of implementing magnetic bead nucleic acid extraction and CRISPR detection using the integrated nucleic acid analysis system in FIG. 9.

### DETAILED DESCRIPTION OF THE INVENTION

The following specific examples will illustrate the implementation methods of the present invention, and those skilled in the art can easily understand the other advantages and effects of the present invention from the content disclosed in this description. The present invention can also be implemented or applied through different specific embodiments, and the details in this specification can be modified based on different perspectives and applications without deviating from the spirit of the present invention. When detailing the embodiments of the present invention, for the ease of explanation, the cross-sectional view representing the device structure will not be regionally enlarged according to the general scale, and the schematic diagram is only an example, which should not limit the scope of protection of the present invention. In addition, in actual implementation, three-dimensional spatial dimensions of length, width, and depth should be properly considered.

For the convenience of description, spatial relationship words such as “below”, “under”, “underneath”, “above”, “on top of”, etc., may be used here to describe the relationship between one component or feature shown in the figure and other components or features. It should be understood that these spatial relationship words are intended to encompass directions other than those depicted in the accompanying drawings for devices in use or operation. Furthermore, when a layer is referred to as “between” two layers, it may be the only layer between the two layers, or there may be one or more layers between them.

In the context of this application, the structure of the first feature described above the second feature may include embodiments in which the first and second features are formed as direct contact, or embodiments in which other features are formed between the first and second features, so that the first and second features may not be in direct contact.

It should be noted that the illustrations provided in this embodiment only illustrate the basic concept of the present invention in a schematic manner. Therefore, only the components related to the present invention are displayed in the diagram, rather than being drawn based on the actual number, shape, or size of the components during an implementation. The type, quantity, or proportion of each component during an actual implementation can be arbitrarily changed, and the layout of its components may also be more complex. To make the illustrations as concise as possible, not all structures are indicated in each diagram.

For the purpose of this disclosure, the word “including” and its variants, such as “comprising” and “consisting of”, should be understood as “including but not limited to”.

In the entire description of this patent, “one embodiment” or “one aspect” refers to the embodiment or aspect comprising specific features, structures, and characteristics that may be included in one embodiment, but may not necessarily be included in all embodiments. In addition, the features, structures, and characteristics disclosed in this patent may be applied in any suitable combination in one or more embodiments.

For the purpose of this disclosure, the term “microfluidics” refers to a device or system capable of manipulating at least one liquid with a cross-sectional size ranging from a few micrometers to about a few millimeters. The term “digital microfluidics” refers to a device or system that can manipulate one or more droplets based on the effects of electrowetting or dielectrophoresis.

For the purpose of this disclosure, “DMF chip”, “DMF device”, and “DMF cartridge” may be used interchangeably, including a first substrate (bottom plate) with a first substrate surface and a second substrate (top cover plate) with a second substrate surface, wherein the second substrate is spaced at a certain distance from the first substrate to define a gap (space between the first and second substrate surfaces), wherein the distance is sufficient to accommodate droplets placed in the gap. Multiple liquid control electrodes are arranged on the surface of the first substrate, and at least some electrodes are covered by a layer of dielectric, and at least a portion of the dielectric layer is hydrophobic. For the purpose of grounding, at least one electrode is provided on the surface of the second substrate, and at least a portion of the electrode is covered by a layer of dielectric, and at least a portion of the dielectric layer is hydrophobic.

For the purpose of this disclosure, the term “droplet” refers to a certain amount of liquid (one liquid or a mixture of different liquids) separated from other parts such as air or other gases, other (usually immiscible) liquids, and solid surfaces (such as the inner surface of a DMF device). The volume range of droplets is large, usually ranging from a few picoliters to several hundred microliters. Droplets can have any shape, including spherical, hemispherical, flattened circular, irregular, etc.

For the purpose of this disclosure, the terms “reservoir” or “liquid reservoir” are used to indicate a portion of a DMF device that can be used to store, retain, and supply liquids, which can be fully enclosed or partially enclosed. A reservoir can be associated with a fluid path that allows liquid to be dispensed into the gaps of DMF devices for droplet operations, or for liquid in other regions of DMF device to enter reservoir for liquid storage or temporary holding.

For the purpose of this disclosure, the terms “filler liquid”, “filler solution” and “filler oil” may be used interchangeably, referring to a liquid that can fully or partially fill the gap of a DMF device, is essentially immiscible with droplets, and does not significantly affect the ability of DMF device electrowetting operations. The filler fluid may contain low viscosity oils, such as silicone oil, mineral oil, paraffin liquid, fluorosilicone oil, etc. The kinematic viscosity of the filler fluid is usually less than 100 cSt (centroStokes), or 50 cSt, or 20 cSt, or 10 cSt, or 5 cSt, or 2 cSt. The filler liquid may contain a small amount of surfactant soluble in the filler liquid, and the volume ratio of the surfactant to the filler liquid is usually within the range of 0.00001% to about 1%, or 0.0001% to about 0.1%, or 0.001% to about 0.01%. Filler fluid can fill the entire or partial gap of a DMF device. A sample (or reagent) and a filler solution can also be placed in the same reservoir on the DMF device, so that when a droplet dispensed from said reservoir is wrapped in a thin layer of filler solution.

Unless otherwise specified, in this disclosure, the term “less than” usually means “equal to or less than”, and “greater than” usually means “equal to or greater than”.

For the purpose of this disclosure, the term “droplet operation” may include droplet dispense/distribution (from a reservoir or continuous fluid flow), movement, merging and mixing, split/segmentation (symmetrically or asymmetrically), shaping (to form a specified shape), suspension and distribution of particles (within a liquid or droplet), etc.

The present invention proposes an integrated nucleic acid analysis system for processing or measuring target analytes in a sample solution and a method for measuring at least one target nucleic acid in the sample. As technical personnel in this field will understand, sample solutions may include but are not limited to body fluids (including but not limited to blood, serum, saliva, urine, etc.), purified samples (such as purified DNA, RNA, proteins, cells, etc.), environmental samples (including but not limited to water, air, agricultural samples, etc.), and biological warfare agent samples. Body fluids can come from any organism. In some implementation schemes, the physical solution may be bodily fluids from mammals, such as bodily fluids from humans.

For the purpose of this disclosure, the terms “target” and “analyte” may be used interchangeably to refer to the analyte or chemical composition being analyzed or tested. An analyte can be an organic or inorganic substance. It can refer to biological molecules (such as proteins, lipids, cytokines, hormones, carbohydrates, etc.), viruses (such as herpesviruses, retroviruses, adenoviruses, lentiviruses), intact cells (including prokaryotic and eukaryotic cells), environmental pollutants (including toxins, insecticides, etc.), drug molecules (such as antibiotics, therapeutic drugs and drug abuse, and drugs), cell nuclei, spores, etc.

For the purpose of this disclosure, the term “antigen” refers to a toxin or other foreign body that can induce an immune response (producing antibodies) in the host organism, especially the production of antibodies.

For the purpose of this disclosure, the term “antibody” refers to a large Y-shaped protein molecule primarily produced by plasma cells, which is used by the immune system to neutralize pathogens, such as pathogenic bacteria or viruses. Antibodies bind specifically to their corresponding antigens. Antibodies can be labeled with another molecule (such as a fluorescent label or enzyme) to facilitate antibody detection or quantitative measurement.

For the purpose of this disclosure, the terms “reagent” and “diagnostic reagent” may be used interchangeably, referring to any material used for reacting with a sample, diluting a sample, mixing a sample, suspending a sample, emulsifying a sample, encapsulating a sample, interacting with a sample, and adding it to a sample.

For the purpose of this disclosure, the terms “freeze-dried reagent” and “lyophilized reagent” may be used interchangeably, referring to reagents prepared using freeze-drying methods, typically used for reagents containing active substances that may deteriorate at elevated temperatures. The preservation of biological reagents is a crucial step in medical diagnostics. From the perspective of reagent preservation, many point of care testing (POCT) reagent kits need to be stored at room temperature, which requires the reagents to undergo dehydration treatment and exist in solid form. To use it in an application, it needs to be dissolved using a buffer solution for subsequent reactions. For example, when liquid reagents drops are put into the liquid nitrogen, they solidify into small balls in a very short period of time. Then, the solid balls are placed in a pre-cooled freeze-drying machine, and the freeze-drying curve is designed to complete the freeze-drying process. The freeze-dried reagent balls can maintain enzyme activity to the maximum extent, and the freeze-dried reagent balls have a loose network structure, allowing for rapid dissolution. Freeze-dried solid balls can be stored and transported at room temperature after packaging, reducing transportation costs and increasing preservation time. The outstanding advantages of freeze-dried bead technology are not only suitable for the production process of nucleic acid reagent POCT technology, but also for the production process of other products that require preservation, ensure active substances, and store and transport biological products at room temperature. In the past few years, this modern technology has been widely utilized.

For the purpose of this disclosure, the terms “magnet” and “magnetic object” may be used interchangeably to refer to objects with a certain shape and magnetism, including samarium cobalt magnets, neodymium iron boron magnets, ferrite magnets, aluminum nickel cobalt magnets, and iron chromium cobalt magnets. Shapes include cylindrical, toroidal, circular, conical, pyramid, and other irregular shapes. Magnets include permanent magnets and non-permanent magnets. Permanent magnets themselves always have magnetism, while non-permanent magnets, such as electromagnets, only exhibit magnetism under certain conditions (such as current passing through).

For the purpose of this disclosure, the term “focusing magnet” is used to refer to a magnet (permanent magnet or electromagnet) with a specific shape, such that the magnetic field on one side is stronger than the magnetic field on the opposite side. Examples include but are not limited to conical magnets or pyramid shaped magnets. The magnetic field at the tip of a conical magnet (or pyramid magnet) is stronger than that of its base.

In the present invention, the term “particle” is used to refer to entities at the micrometer or nanometer level, which can be natural or artificially made, such as cells, subcellular components, liposomes, viruses, nanospheres, and microspheres, or smaller entities such as biomolecules, proteins, DNA, RNA, etc. It can also refer to liquid droplets that are not fused with suspension media. It can also refer to small bubbles in liquids, etc. The (linear) size of particles can range from a few nanometers to several hundred micrometers.

In the present invention, the term “bead” can refer to any bead or particle that reacts with a solution. Beads can be of any different shape, such as spherical, egg shaped, cubic, disc-shaped, or irregular. Beads can be placed inside droplets, on the inner surface of DMF devices, in the filler solution of DMF devices, in liquid reservoirs, etc. Beads can be made from various materials, such as resin, polymer, glass, nanomaterials, etc., and can have arbitrary size such as microbeads and nanobeads. Beads can have magnetic responsiveness, in which case at least one or part of their components are composed of magnetic responsive materials, while the remaining materials may contain polymeric materials, coatings, or groups linked to detection reagents. Examples of beads include quantum dots, polyethylene microspheres, silica microspheres, fluorescent microspheres or nanospheres, magnetic microspheres, magnetic nanobeads, flow cytometry microspheres, etc.

For the purpose of this disclosure, the term “magnetic bead” refers to beads containing magnetic responsive materials. Examples of magnetic responsive materials include ferromagnetic materials, paramagnetic materials, supermagnetic materials, and ferrous magnetic materials. Examples of paramagnetic materials include metals such as nickel, iron, and cobalt, as well as metal oxides such as Fe3O4, Cr2O3, NiO, Mn2O3, etc. Magnetic responsive materials can constitute the entire magnetic bead, a part of the magnetic bead, or a certain component of the magnetic bead. The remaining parts of the magnetic bead can include polymer materials and coating parts that allow the attachment of target particles. Magnetic beads can be used in various measurements, among which magnetic beads are usually used to bind one or more target substances in a mixture, such as analytes or pollutants. Measurement usually requires an effective magnetic bead cleaning process to reduce the amount of one or more substances that may come into contact with the surface of magnetic beads in droplets containing magnetic beads.

Validation experiments based on digital microfluidics have shown that magnetic beads coated with antihuman serum albumin antibodies can be used for separating human serum albumin. The validation experiment of extracting DNA from whole blood samples using magnetic beads has also been implemented on a digital microfluidic platform. Implementing DNA extraction on a digital microfluidic platform is similar to traditional methods, typically requiring a cell lysis pre-treatment step.

For the purpose of this disclosure, the terms “fixed” or “focused” may be used interchangeably to indicate that magnetic beads are essentially confined to a specified position in droplets, reservoirs, or filler liquids on DMF devices. For example, in one implementation, fixed magnetic beads are essentially confined to a location within the droplet to allow for droplet separation operations, resulting in one droplet with essentially no magnetic beads and one droplet with a majority of the magnetic beads (residual liquid).

For the purpose of this disclosure, the terms “magnetic bead washing” and “magnetic bead cleaning” may be used interchangeably to indicate the use of liquids to reduce the quantity or concentration of a certain (or several) substance on the magnetic beads. The decrease in substance quality or concentration can be partial or complete. This substance can be any one of multiple substances, such as the composition of the sample, pollutants, and excess reagents, etc.

For the purpose of this disclosure, “magnetic bead operation” may consist of the following operations or any combination thereof:

1. To group (or focus)—To group of magnetic beads within droplets, within reservoirs, and between droplets (or reservoirs) on DMF devices. The size of the grouped magnetic beads is less than 10 millimeters, or less than 5 millimeters, or less than 2 millimeters. It should be pointed out that the grouped magnetic beads typically have clear boundaries, but the boundaries can also be quite blurry. The proportion of grouped magnetic beads exceeds 30%, or 50%, or 70%, or 80%, or 90% of all specific magnetic beads.

2. To Immobilize (or fix)—To keep the magnetic beads at the designated position on the DMF device for a specified period of time. During this period of time, operations such as droplet controls can be performed.

3. To transport—To move magnetic beads from one position to another on a DMF device, including but not limited to moving magnetic beads from one droplet/reservoir to another, and moving between a droplet and a reservoir.

4. To disperse (or suspend)—By removing (or other controlling) the magnetic field, the accumulated (grouped or focused) magnetic beads can be dispersed in droplets or reservoirs, which can be carried out simultaneously with droplet operations.

For the purpose of this disclosure, “amplification” refers to the process of increasing the quantity or concentration of the analyte under test. Non-limiting examples include polymerase chain reaction (PCR) and its variants (such as quantitative competitive PCR, immunoPCR, reverse transcription PCR, etc.), strand displacement amplification (SDA), nucleic acid sequence based amplification (NASBA), loop mediated isothermal amplification (LAMP), Helicase dependent amplification (HAD), etc.

For the purpose of this disclosure, the terms “layer” and “film” may be used interchangeably to refer to the structure of the subject, which is typically but not necessarily planar or substantially planar, and is typically deposited, formed, coated, or otherwise placed on another structure.

For the purpose of this disclosure, the term “ground” (as used for “ground electrode” or “ground voltage”) refers to the voltage of the corresponding electrode being zero or sufficiently close to zero. All other voltage values, although typically less than 300 volts in amplitude, should be high enough to fully observe the electrowetting or electrophoresis effect.

It should be pointed out that when arranging a covered dielectric layer, the space between adjacent electrodes in the same layer is usually filled with dielectric materials. These spaces can also be empty or filled with gases such as air, nitrogen, helium, and argon. All electrodes in the same layer and electrodes in different layers are preferably electrically insulated.

As used in this article, the term “contact angle” refers to the angle formed when the liquid vapor interface comes into contact with a solid surface. When the three-phase liquid phase, solid phase, and gas phase (which can be a mixture of atmospheric and liquid equilibrium concentrations of vapor) reach thermodynamic equilibrium, the shape of the liquid gas interface is determined by the Young Laplace equation.

$${γ}_{SG} - {γ}_{SL} - {γ}_{LG} cos {θ}_{c} = 0$$

where γ_{SG} denotes the solid-vapor interfacial energy, γ_{SL} denotes the solid-liquid interfacial energy, γ_{LG} denotes the liquid-vapor interfacial energy (i.e., the surface tension), and θ_{c} denotes the equilibrium contact angle. The following is a schematic of a liquid droplet showing the quantities in the Young-Laplace equation. It should be pointed out that the equation also works if the gas phase is replaced by another immiscible liquid phase.

In a pure liquid, each molecule in the bulk is pulled equally in every direction by neighboring liquid molecules, resulting in a net force of zero. However, the molecules exposed at the surface do not have neighboring molecules in all directions to provide a balanced net force. Instead, they are pulled inward by the neighboring molecules, creating an internal pressure. As a result, the liquid contracts its surface area to maintain the lowest surface free energy. This intermolecular force, i.e., liquid-vapor interfacial energy γ_{LG}, to contract the surface is called the surface tension, and it is responsible for the shape of liquid droplets. In practice, external forces such as gravity deform the droplet; consequently, the contact angle is determined by the combination of surface tension and external forces (typically gravity). The contact angle is expected to be characteristic for a given solid-liquid system in a specific environment.

A Hydrophobic surface has the characteristic of repelling liquids, while a hydrophilic surface has the characteristic of attracting liquids. For the purpose of this disclosure, a “hydrophobic surface” has a contact angle greater than 90°, while a hydrophilic surface has a contact angle less than 90°.

For the purpose of this disclosure, it can be understood that when any form of liquid (such as droplets or continuum, which may be in motion or stationary) is described as being “on”, “at” or “above” the electrode, array, matrix, and surface, the liquid may come into direct contact with the electrode, array, matrix, and surface, or may come into contact with one or more layers or membranes inserted between the liquid and the electrode, array, matrix, and surface.

For the purpose of this disclosure, it can be understood that when a given component such as a layer, region, and substrate is referred to as placed or formed on another component “on”, “in”, and “at”, the given component may be directly located on that other component, or alternatively, there may be intermediate components (such as one or more buffer layers, interlayers, and electrodes). It can also be understood that the terms “placed on” and “formed on” can be used interchangeably to describe how a given component is positioned or positioned relative to another component. Therefore, the terms “placed on” and “formed on” are not intended to introduce any limitations on specific methods of material transfer, deposition, and fabrication.

For the purpose of this disclosure, the terms “Printed Circuit Board (PCB)” or “Print Circuit Board” may be used interchangeably, referring to a circuit board without soldered components, mainly composed of the following parts:

1. Circuit and Pattern: The material used in a circuit is usually copper, which can provide a conductive path between electronic components. In addition, a large copper surface is usually designed as the grounding and power layer. The circuit and the diagram are made simultaneously.

2. Dielectric layer: used to maintain the insulation between the circuit and its layers, also known as substrate.

3. Through hole (or Via): A through hole allows two or more layers of circuits to be connected to each other, while larger through holes are used as part inserts. Additionally, non through holes are commonly used for surface mount positioning.

4. Solder Mask: Not all copper surfaces need to be coated with tin, so non tin areas will be printed with a layer of material (usually epoxy resin) to isolate the copper surface from tin, avoiding short circuits between non tin lines. According to different processes, it is divided into green oil, red oil, and blue oil.

5. Silk screen: This is a non-essential component, and its main function is to mark the names and location boxes of each component on the circuit board for easy maintenance and identification after assembly.

For the purpose of this disclosure, the terms “testing”, “detection”, and “measurement” may be used interchangeably for the process of obtaining physical quantities (such as position, charge, temperature, concentration, pH value, brightness, fluorescence, etc.). In general, at least one sensor (or detector) is used to obtain physical quantities and convert them into signals or information that can be recognized by humans or instruments. There can be other components between the object to be tested and the sensor, such as lenses, reflectors, filters, etc. used in optical measurements, and resistors, capacitors, transistors, etc. used in electrical measurements. Moreover, in order to make measurement possible or easier, other auxiliary devices or devices are often used in the measurement. For example, light sources such as Lasers or Laser diodes are used to excite particles from the electronic ground state to the electronic excited state. When the excited state particles return to the ground state, they sometimes emit fluorescence, and measuring the fluorescence intensity at this time can be used to measure the concentration of a certain particle in a liquid sample. Optical sensors include CCD, photodiodes, photomultiplier tubes, etc. In terms of electricity, there are operational amplifiers, analog-to-digital converters, thermocouples, thermistors, etc.

Measurement can be performed on multiple parameters in multiple samples simultaneously or in a certain order. For example, when measuring the fluorescence of a certain particle in a droplet using a photodiode, the position of the droplet can also be obtained simultaneously through capacitance measurement. Sensors or detectors are usually connected to the Central Processing Unit (CPU) or computer, which runs corresponding software to analyze the measured signal and usually converts it into information that can be read by humans or other devices. For example, measuring and analyzing the fluorescence intensity of a particle in a liquid can be used to infer the concentration of that particle. As a non-limiting example, optical measurements include laser induced fluorescence measurement, infrared spectroscopy, Raman spectroscopy, chemiluminescence measurement, surface plasmon resonance measurement, absorption spectroscopy, etc.; Electrical measurements include amperometry, voltammetry, photoelectrochemistry, Coulomb analysis, capacitance measurement, and AC impedance measurement.

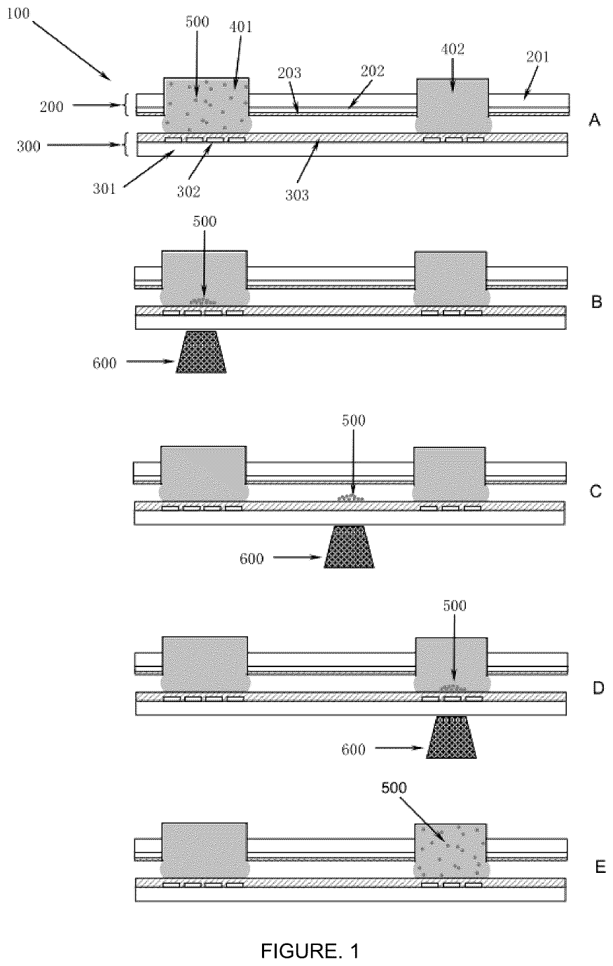

The following is a specific description of the implementation scheme for processing biological samples in the present invention. For ease of explanation, the corresponding drawings (FIGS. 1A to 10) will be mentioned when needed. It should be noted that the purpose of these examples is to help illustrate, not to limit the will and spirit of invention.

The accompanying drawings and specific descriptions in this article further demonstrate and interpret the principles disclosed in this invention, and enable technical personnel in the relevant field to manufacture and use corresponding instruments, DMF devices, and described methods.

For the purpose of this disclosure, some or all functional modules (such as magnet control module, temperature control module, optical measurement module, etc.) can be automatically controlled. Programs (software or firmware) running on microprocessors or computers are typically used to achieve automatic control.

FIGS. 1A-1E show a side view of a portion of the DMF device in an integrated nucleic acid analysis system of the present invention, overall labeled 100, as a preferred embodiment for implementing magnetic bead control. FIG. 1A shows the different reservoirs of liquid 401 and liquid 402 on the DMF device. The bottom plate 300 includes droplet control electrodes 302 and a dielectric layer 303 deposited on the substrate 301. The top cover plate 200 includes a grounding electrode 202 and a dielectric layer 203 on the deposited substrate 201. It should be pointed out that the DMF device structure shown here is for the purpose of explaining magnetic bead manipulation and does not represent all possibilities. In some embodiments, DMF devices can be implemented in various different ways. For example, 1) control electrodes can have different shapes, such as rectangular, square, trapezoidal, pentagonal, hexagonal, and irregular shapes, and can be arranged and combined in a straight line or other shapes; 2) The control electrodes can be placed in different layers (usually electrically insulated from each other), as described in patent WO 2008/147568 titled “Electrowetting Based Digital Microfluidics” (invented by Wu Chuanyong); 3) The dielectric layer can also have two or more layers, and the materials used include paraxylene C, silicon nitride, silicon dioxide, tantalum oxide, etc.; One of the layers can be hydrophobic materials, such as Teflon, Cytop, and FluoroPel.

The substrate can be any non-conductive material or conductive material coated with a non-conductive layer, as long as it has sufficient mechanical strength to maintain its shape within the required system operation and storage conditions. In terms of transparency, it can be transparent, semi-transparent, and opaque. Transparent substrates can be made of various transparent materials, such as glass, quartz, plastic, transparent ceramics, transparent printed circuit boards, etc. Electrodes can be made of any conductive material, such as metals, alloys, and conductive polymers. It can be made from one material or a mixture of different materials. The transparent electrodes on DMF devices can be made of transparent conductive materials (such as indium tin oxide (ITO), aluminum doped zinc oxide (AZO), transparent conductive polymers (polyacetylene, polyaniline, etc.), or transparent nanomaterials.

The voltage control module is used to provide voltage control signals to the droplet control electrode. It usually has multiple outputs, with a maximum quantity of 1000000, or 100000, or 10000, or 1000. The voltage output can be unipolar or bipolar, with a voltage amplitude of less than 1000 volts, less than 300 volts, less than 100 volts, less than 60 volts, or less than 30 volts. AC or DC signals with a voltage frequency less than 10 MHZ (megahertz), or less than 1 MHZ, or less than 100 KHz (kilohertz), or less than 20 KHz, or less than 5 KHz, or less than 1 KHz. The waveform of voltage can be square wave, sine wave, sawtooth shape, pulse width modulation signal, etc. The voltage control module is usually programmed by the microprocessor or computer on the circuit board through SPI (Serial Peripheral Interface), I2C (Inter-Integrated Circuit), USB (Universal Serial Bus), parallel port, Ethernet, Wi-Fi, or Bluetooth to program the order, duration, amplitude, and frequency of the output signal. Spring loaded electrical contact pins or connector pads can be used to transmit multiple high-voltage control signals to the electrodes of a DMF device.

In FIG. 1A, the magnetic beads 500 are uniformly distributed in reservoir 401 of the DMF device. In FIG. 1B, place the magnet 600 tightly against the DMF device in the position of reservoir 401. Under the action of the magnetic field generated by the magnet, the magnetic bead 500 is gathered at the bottom of reservoir 401. As shown in FIGS. 1C and 1D, the magnet moves the magnetic beads to reservoir 402. In FIG. 1E, the magnet is moved away from the DMF device, and the magnetic beads are resuspended and dispersed in reservoir 402. Magnetic beads suspended and dispersed in the liquid in reservoir 402 may require droplet movement or other means of assistance.

The gap between the reservoirs on a DMF device may be surrounded by air or filler liquid. FIG. 1A-1E shows the process of magnetic beads being moved from inside a reservoir through a medium (air or filler liquid) to another reservoir. As shown in the figure, the device is hydrophobic to the droplets. The droplets on the electrode can be manipulated (moved, divided, merged, mixed, etc.) through the electrowetting effect.

The phrases used in this article, such as “the magnet is moved (or brought in, transported) to approach” and “the magnet is moved close to”, are intended to refer to the relative position of the magnet and DMF device. The magnetic force generated by the magnet has a significant effect on the magnetic beads in the DMF device. On the contrary, phrases such as “magnet removed” and “magnet moved away” are intended to indicate that the magnet has no or negligible effect on the magnetic beads in the device.

Depending on different measurements, the movement speed of a magnet can usually be controlled by a motor, and different applications require different magnet speeds, typically ranging from 0.1 to 100 mm/second, or 0.5 to 20 mm/second, or 1 to 10 mm/second.

In the above embodiment, the magnet contacts the substrate and moves along the bottom of the DMF device. In some embodiments, the magnet may also be located at the top of the DMF device; Or a pair of magnets, one above and the other below the DMF device, used to manipulate magnetic beads in said DMF device.

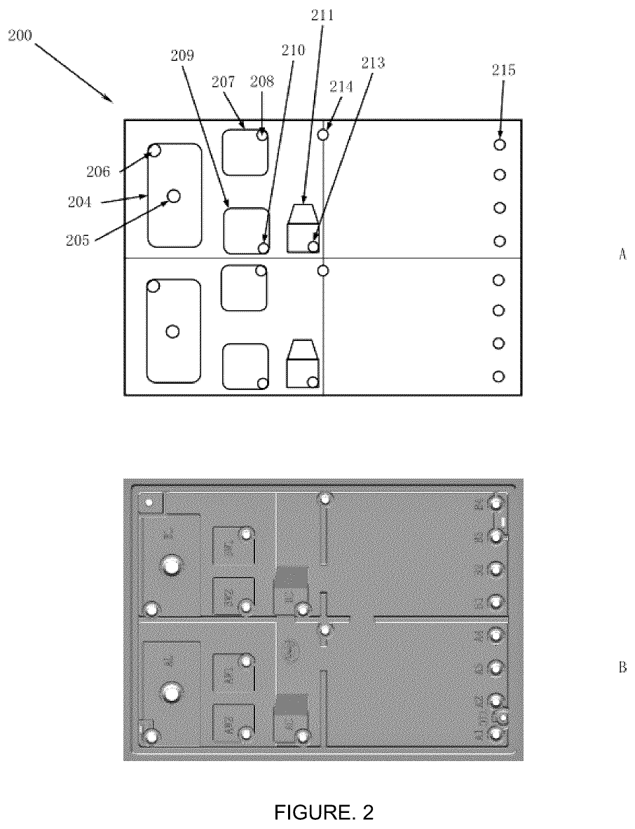

FIG. 2A shows a top view of a DMF device 200 in the present invention, which is generally represented as 200 and can run and analyze two samples simultaneously. Both the bottom plate and top cover plate have two areas with the same function, which can be used to process two samples separately. Reservoir 204 can be used for sample lysis and nucleic acid capture, while the addition ports 205 and 206 are used for loading samples, lysis solution, and magnetic bead solution for nucleic acid extraction. Reservoirs 207 and 209 are used for nucleic acid cleaning. Add liquid holes 208 and 210 for loading magnetic bead cleaning solution. Reservoir 211 is used for nucleic acid elution, and the liquid loading port 213 is used to load the eluent. Addition port 215 is used to load reagents for nucleic acid amplification and optical measurement. Addition port 214 can be used to load reagents for negative or positive controls.

For further clarification, a simplified measurement procedure is provided here, which includes: 1) Bring the magnet to make contact with the DMF device at position 204, moving the magnet along a specified trajectory within the range of reservoir 204, and gathering magnetic beads with captured nucleic acids at the bottom of the reservoir; 2) Use a magnet to move the grouped magnetic beads to reservoir 207 and clean the magnetic beads inside the reservoir; 3) Move the focused magnetic beads to reservoir 209 and perform another cleaning inside the reservoir; 4) Move the washed magnetic beads to reservoir 211 and perform nucleic acid elution; 5) Using an electrowetting electrode, disperse one or more droplets of the eluent containing nucleic acid from the elution reservoir and move them to the right side, mixing them with PCR reagents loaded through loading port 215; 6) Perform thermal amplification and optical measurements.

FIG. 2B is a top view of an actual physical design of the top cover plate of the DMF device based on the present invention. The material of the top cover plate is polycarbonate, which is produced using injection molding technology. The AL and BL reservoirs were used for magnetic bead based extraction of nucleic acids from two samples (A and B), respectively; AE1 and AE2 reservoirs are used for magnetic bead cleaning of sample A, while BE1 and BE2 reservoirs are used for magnetic bead cleaning of sample B; The AE and BE reservoirs are used for nucleic acid elution of samples A and B. A1-A4 wells are used to add reagents for analyzing sample A, and B1-B4 wells are used to add reagents for analyzing sample B; The OIL hole is used to add filler fluid to the DMF device.

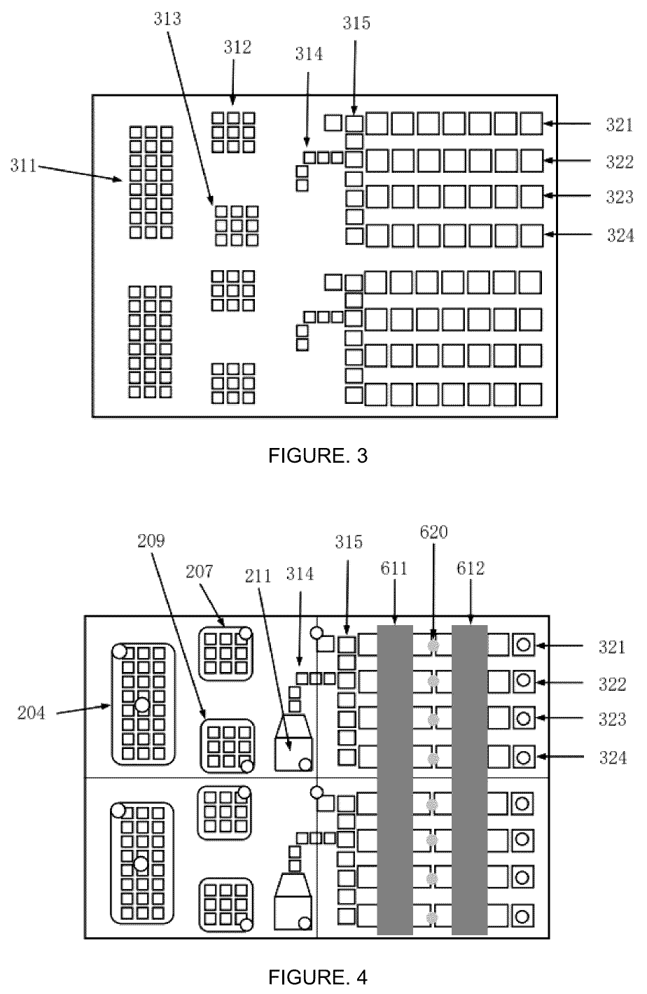

FIG. 3 shows the corresponding electrowetting electrode layout for the DMF device shown in FIG. 2A. Optional electrodes 311 can be used to assist in the mixing of samples with lysis buffer and magnetic beads, optional electrodes 312 and 313 can be used to assist in the washing of magnetic beads, electrodes 314 and 315 can be used to separate droplets from the elution reservoir and move them to the amplification reaction area, and electrodes 321 to 324 can be used to move (including back and forth repeated movement) droplets to achieve amplification and/or measurement.

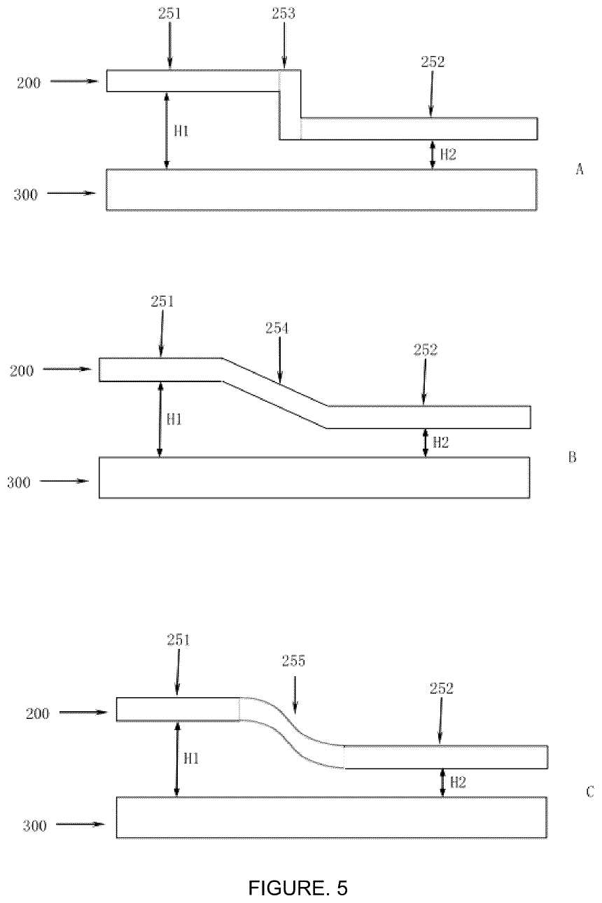

FIG. 4 is a functional schematic diagram combining FIG. 2A and FIG. 3. FIG. 4 shows the temperature control areas 611 and 612, as well as the optical detection point 620. In one embodiment, both optical excitation and detection are accomplished through the substrate of the DMF device. Therefore, this part of the DMF device substrate needs to be optically transparent (including the substrate, electrode, and dielectric layer). In the present invention, optical measurement is carried out within the visible light range, with wavelengths typically between 350-750 nm. Higher light transmittance means higher system detection sensitivity. The transmittance of the bottom plate optical measurement part of DMF devices is usually greater than 30%, or greater than 50%, or greater than 70%, or greater than 90%.

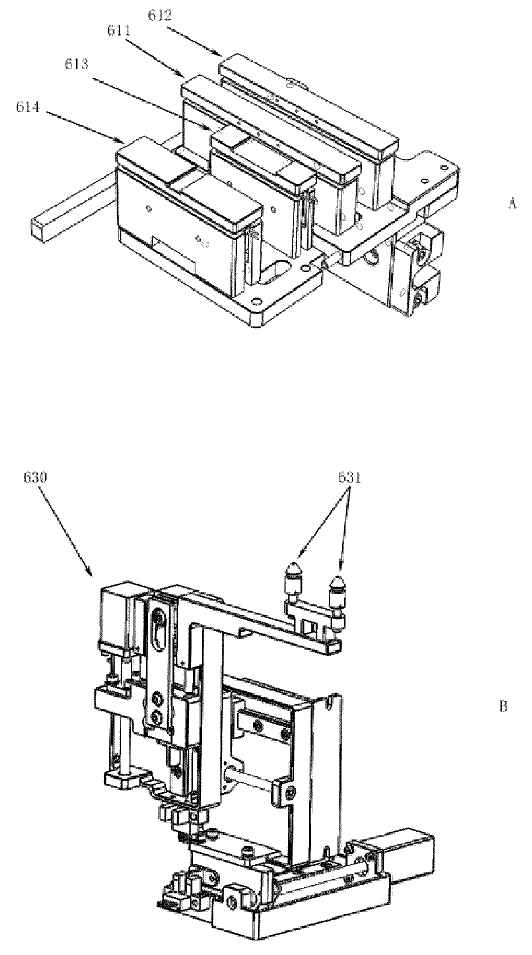

In a DMF device, in order to facilitate droplet dispensing or droplet control, it is sometimes necessary to design the gap of a DMF device with different heights at different functional positions. FIG. 5A-5C shows a side view of the DMF device where the gap height changes from H1 to a smaller value H2. FIGS. 5A and 5B are two common designs, and FIG. 5C is a design proposed by the present invention. In FIG. 5A, the gap height changes from H1 in region 251 to H2 in region 252 at a single point 253; In FIG. 5B, the gap height linearly changes from H1 in region 251 to H2 in region 252 in region 254, with the corresponding slope being a constant.

In mathematical terms, the slope at a point on a curve is the slope of the tangent at that point. In FIG. 5A-5C, the slope is zero at regions 251 and 252. In FIG. 5A, the slope is infinite at point 253. In FIG. 5B, the slope is a constant throughout region 254, which can be calculated by the gap height difference and region length. It is equal to (H1-H2) divided by the length of region 254. For example, if H1 is 2.5 mm, H2 is 0.5 mm, and the length of region 254 is 10 mm, the absolute value of the slope is 0.2.

In the present invention, FIG. 5C is proposed, where the gap height changes from H1 in region 251 to H2 in region 252 in region 255. In region 255, the slope (absolute value) continuously increases from zero to a finite value, and then continuously decreases to zero. As a comparison, in FIG. 5A, the slope changes from zero to infinity at position 253 and then back to zero; In FIG. 5B, the slope changes directly from zero to a non-zero value, and then returns directly to zero. In other words, the slope of the lower surface of the top cover plate at the gap change of the DMF device in FIGS. 5A and 5B will undergo a non-continuous change, while in FIG. 5C, the corresponding slope change is continuous. Without further involving the mathematical details of liquid behavior, FIG. 5C proposes a design that makes it easier for droplets to transition from one gap height to another. Specifically, this makes it easier to dispense droplets from the liquid reservoir.

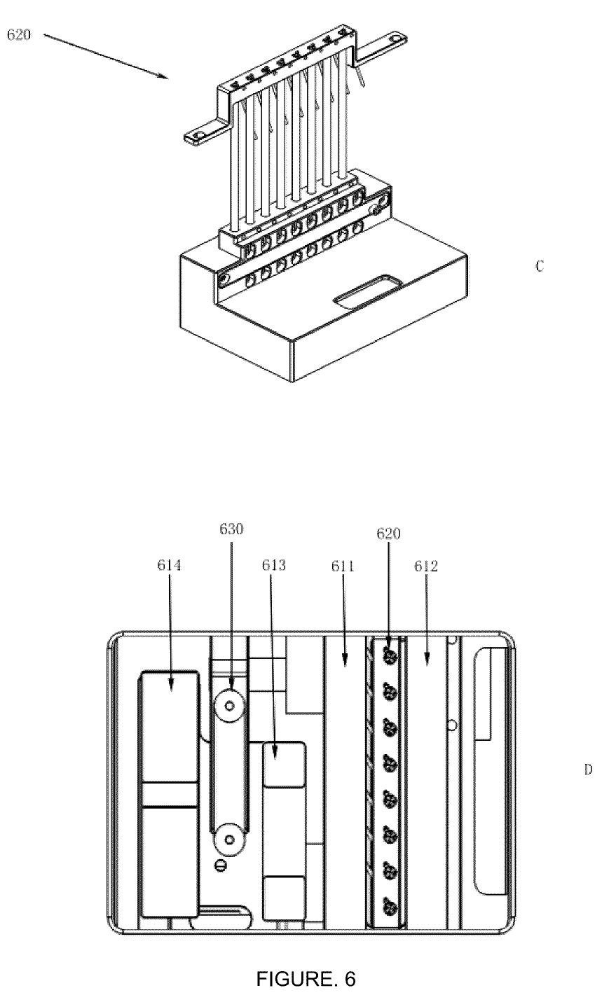

FIG. 6A shows an implementation of a temperature control module. Temperature control modules 611 and 612 are used for nucleic acid amplification. The temperature control module 613 can be optionally used to assist in nucleic acid elution, while the temperature control module 614 can be optionally used to assist in nucleic acid extraction. FIG. 6B shows the magnet control module 630. FIG. 631 shows two magnets used to manipulate magnetic beads in DMF devices. FIG. 6C shows the design of the optical detection module. FIG. 6D shows a top view of temperature control modules 611 to 614, magnet 630, and optical detection module 620.

From FIGS. 6A to 6D, it can be seen that the movement range of the magnet is limited when the temperature control modules (especially 613 and 614) are physically close to the DMF device. Due to the fact that not all functional modules are required simultaneously during the experiment, some modules can be moved away so that other modules can have more space to move around when needed. Therefore, in one embodiment, the instrument is designed to move one or more temperature control elements away from near or in contact with the DMF device when temperature control is not required, thereby allowing the magnet to move close to the expected position of the DMF device. In another embodiment, when the magnet 630 is not in use, it can be moved to one end of the DMF device to allow temperature control modules 613 and 614 to move closer to or in contact with the DMF device. In another embodiment, magnet 630 can be held in one or more spaces between temperature control modules 613 and 614, allowing temperature control modules 613 and 614 to be moved close to or in contact with DMF devices.

The magnet and heating block mentioned earlier are independent devices. In practical applications, the two devices can be integrated together, which means that one device can be used to control the temperature of DMF devices and generate magnetism to control the magnetic beads in DMF devices. Using this integrated module can save space and simplify instrument design. Due to the simplicity of the relevant design, specific example descriptions will not be provided here.

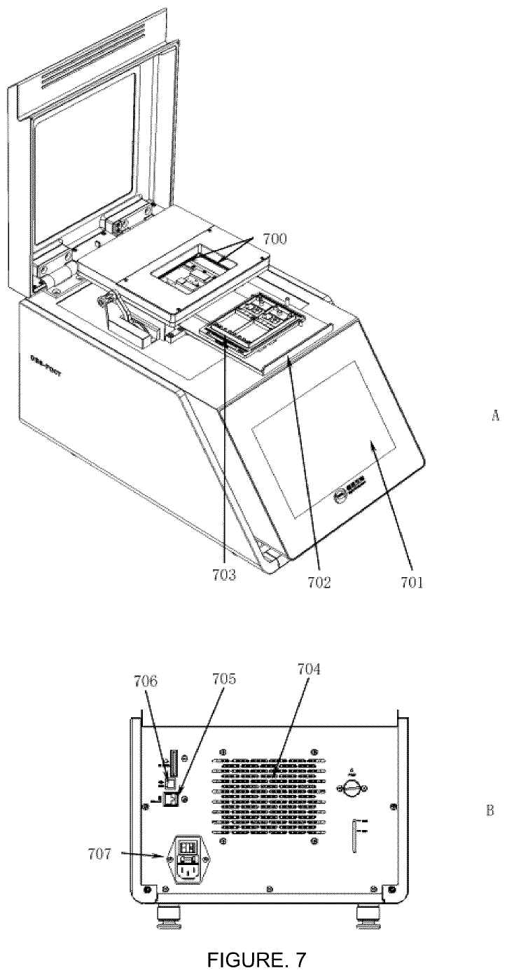

FIGS. 7A and 7B show a preferred embodiment of an integrated nucleic acid analysis system. FIG. 7A is the top front side view of the device, and FIG. 7B is the rear view. 700 is a spring probe that can be used as an interface for droplet control between instruments and DMF devices, providing droplet control voltage to the electrowetting electrodes on DMF devices. 701 is a touch screen designed to provide a user-friendly graphical interface for users to input commands and display help, experimental status, and measurement results. 702 is a sliding tray used for loading and unloading DMF devices. When pushed in, the spring probe 700 contacts the bottom substrate of the DMF device. 703 is a DMF device. In FIG. 7B, 704 is a ventilation port (usually equipped with a fan inside) used to discharge the heat generated by the instrument during operation. 705 is a USB port, while 706 is an Ethernet port. They can optionally provide a way for application software running on a computer to communicate with the instrument, such as sending commands or receiving data. 707 is an AC power port.

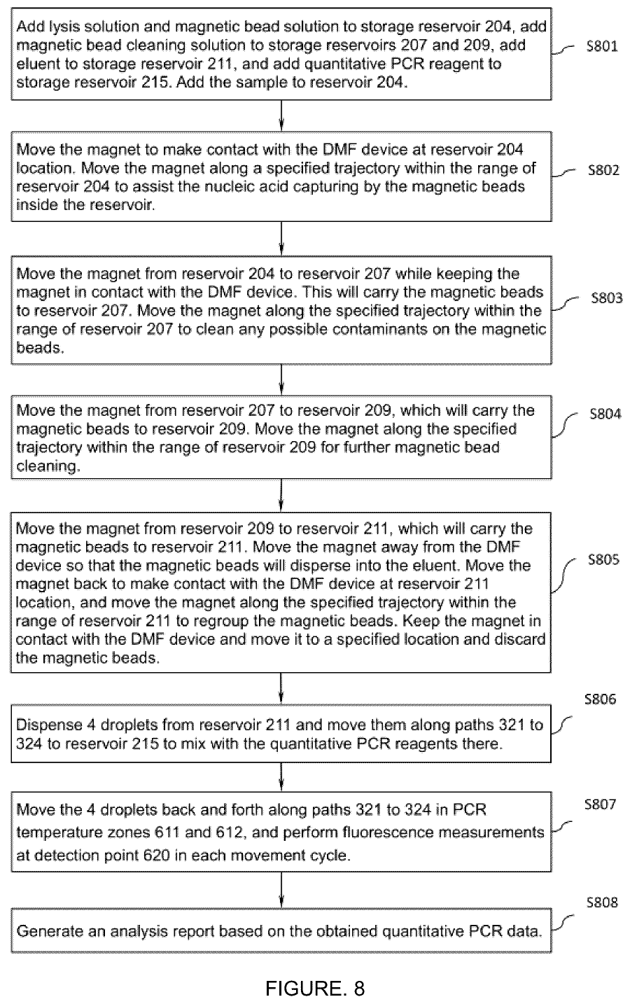

FIG. 8 shows an example of using the nucleic acid analysis system in previous Figures for magnetic bead nucleic acid extraction and quantitative PCR analysis.

In step S801, add lysis solution and magnetic bead solution to reservoir 204, add magnetic bead cleaning solution to reservoirs 207 and 209, add eluent to reservoir 211, and add quantitative PCR reagent to reservoir 215. Add the sample to reservoir 204.

In step S802, move the magnet to contact the DMF device at reservoir 204 location. Move the magnet along a specified trajectory within the range of reservoir 204 to assist the capturing of the nucleic acid molecules by the magnetic beads inside the reservoir.

In step S803, move the magnet from reservoir 204 to reservoir 207 while keeping the magnet in contact with the DMF device. This will carry the magnetic beads to reservoir 207. Move the magnet along the specified trajectory within the range of reservoir 207 to clean any possible contaminants on the magnetic beads.

In step S804, move the magnet from reservoir 207 to reservoir 209, which will carry the magnetic beads to reservoir 209. Move the magnet along the specified trajectory within the range of reservoir 209 for further magnetic bead cleaning.

In step S805, move the magnet from reservoir 209 to reservoir 211, which will carry the magnetic beads to reservoir 211. Move the magnet away from the DMF device so that the magnetic beads will disperse into the eluent. Move the magnet back to contact the DMF device at reservoir 211 location, and move the magnet along the specified trajectory within the range of reservoir 211 to regroup the magnetic beads. Keep the magnet in contact with the DMF device and move it to a specified location and discard the magnetic beads.

In step S806, dispense 4 droplets from reservoir 211 and move them along paths 321 to 324 to reservoir 215 to mix with the quantitative PCR reagents there.

In step S807, move the 4 droplets back and forth along paths 321 to 324 in PCR temperature zones 611 and 612, and perform fluorescence measurements at detection point 620 in each movement cycle.

In step S808, generate an analysis report based on the obtained quantitative PCR data.

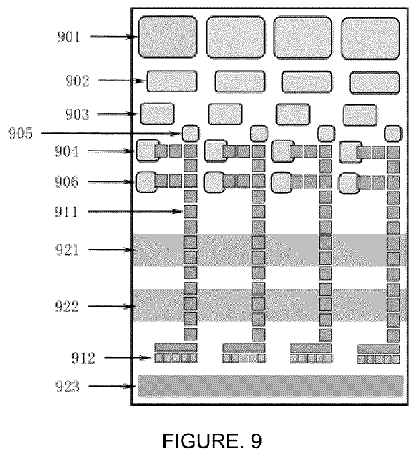

FIG. 9 shows a top view of a DMF device and instrument module in another example of the present invention, which can run and analyze four samples simultaneously. Reservoir 901 can be used for sample lysis and nucleic acid capture, reservoirs 902 and 903 for nucleic acid cleaning, reservoir 904 for nucleic acid elution, reservoir 905 for loading PCR reagents, and reservoir 906 for loading Cas reagents. Dark squares 911 are electrowetting electrodes used for manipulating liquids in the DMF device. Each small square in 912 has a solid (air dried) gRNA reagent for detecting (potentially different) nucleic acid molecules in the reactants. 921, 922, 923 are three independent temperature control modules in the instrument, which can control the corresponding chip area at different temperatures.

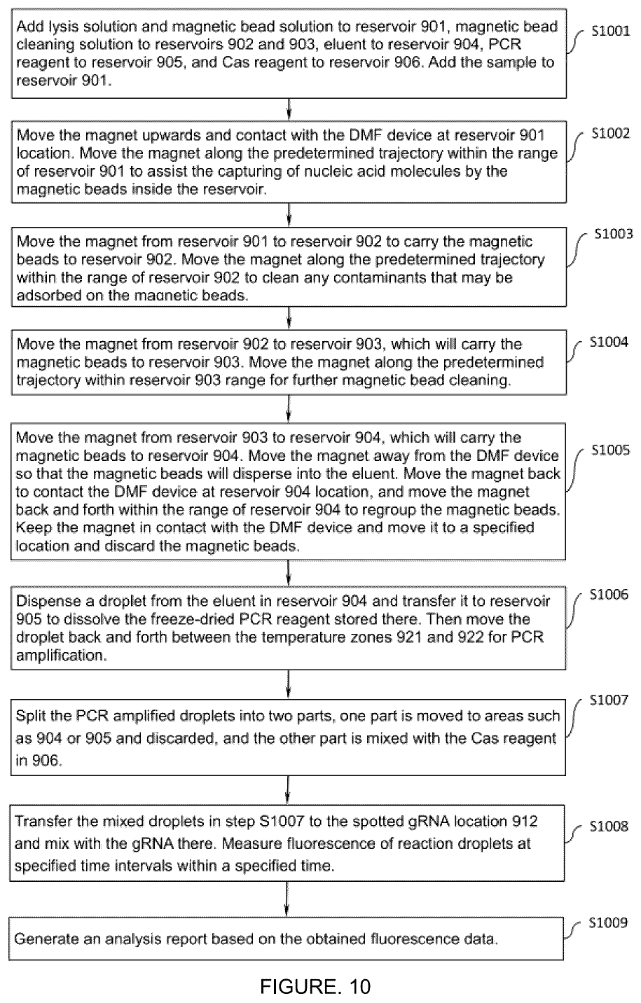

FIG. 10 shows an example of using the integrated nucleic acid analysis system provided in FIG. 9 for magnetic bead nucleic acid extraction and CRISPR detection.

In step S1001, add lysis solution and magnetic bead solution to reservoir 901, magnetic bead cleaning solution to reservoirs 902 and 903, eluent to reservoir 904, PCR reagent to reservoir 905, and Cas reagent to reservoir 906. Add the sample to reservoir 901.

In step S1002, move the magnet upwards and contact the DMF device at reservoir 901 location. Move the magnet along the predetermined trajectory within the range of reservoir 901 to assist the capturing of nucleic acid molecules by the magnetic beads inside the reservoir.

In step S1003, move the magnet from reservoir 901 to reservoir 902 while keeping the magnet in contact with the DMF device. This will carry the magnetic beads to reservoir 902. Move the magnet along the predetermined trajectory within the range of reservoir 902 to clean any contaminants that may be absorbed on the magnetic beads.

In step S1004, move the magnet from reservoir 902 to reservoir 903, which will carry the magnetic beads to reservoir 903. Move the magnet along the predetermined trajectory within reservoir 903 range for further magnetic bead cleaning.

In step S1005, move the magnet from reservoir 903 to reservoir 904, which will carry the magnetic beads to reservoir 904. Move the magnet away from the DMF device so that the magnetic beads will disperse into the eluent. Move the magnet back to contact the DMF device at reservoir 904 location, and move the magnet back and forth within the range of reservoir 904 to regroup the magnetic beads. Keep the magnet in contact with the DMF device and move it to a specified location and discard the magnetic beads.

In step S1006, dispense a droplet from the eluent in reservoir 904 and transfer it to reservoir 905 to dissolve the freeze-dried PCR reagent stored there. Then move the droplet back and forth between the temperature zones 921 and 922 for PCR amplification.

In step S1007, split the PCR amplified droplets into two parts, one part is moved to areas such as 904 or 905 and discarded, and the other part is mixed with the Cas reagent in 906.

In step S1008, transfer the mixed droplets in step S1007 to the spotted gRNA location 912 and mix with the gRNA there. At this point, Cas binds to the gRNA, and after finding the corresponding DNA molecule, the nucleic acid molecules (including reporter molecules) in the droplets are cleaved. Measure fluorescence of reaction droplets at specified time intervals within a specified time.

In step S1009, generate an analysis report based on the obtained fluorescence data.

It should be mentioned here that reagents can be pre-stored on DMF devices, and users only need to load samples. This makes the device easier to operate and reduces the chance of contamination (or cross contamination) during testing.

It should be pointed out that the above examples and advantages are for illustrative purposes and are not exhaustive.

Although preferred embodiments of the present invention have been shown and described, it should be understood that various changes can be made without departing from the spirit and scope of the present invention.

The above embodiments are only illustrative of the principle and efficacy of the present invention, and are not intended to limit the present invention. Anyone familiar with this technology may modify the above embodiments without violating the spirit or scope of the present invention. Therefore, all equivalent modifications or changes completed by those with ordinary knowledge in the technical field without departing from the spirit and technical ideas disclosed in the present invention should still be covered by the claims of the present invention.

1. An integrated nucleic acid analysis system, comprising:
at least one digital microfluidics (DMF) device for receiving at least one sample;
wherein said at least one DMF device comprises:
a bottom plate with a first surface, wherein a first set of electrowetting electrodes for droplet or liquid manipulations is deposited on said bottom plate, said first surface is substantially flat, and at least a portion of said bottom plate is optically transparent;
a top plate, comprising a second surface parallel to said first surface and mated thereto to form a gap for liquid operations, wherein said gap comprises a first reservoir for nucleic acid extraction and a third reservoir for nucleic acid elution, and said top plate comprises fluid holes for loading or unloading of sample or reagent;
an instrument, for performing a set of operations on a fluid sample in said at least one DMF device, wherein said set of operations comprises nucleic acid extraction, purification, thermal amplification, and optical measurement.

2. Said integrated nucleic acid analysis system according to claim 1, wherein said at least one DMF device further comprises a second reservoir for nucleic acid cleaning and/or a fourth reservoir for magnetic bead cleaning.

3. Said integrated nucleic acid analysis system according to claim 1, wherein said bottom plate of said at least one DMF device further comprises a second set of electrowetting electrodes to form pathways for nucleic acid amplification.

4. Said integrated nucleic acid system according to claim 1, wherein a height of said gap of said at least one DMF device varies, and a corresponding slope change on the second surface is continuous.

5. Said integrated nucleic acid analysis system according to claim 1, wherein a transmittance of said at least a portion of said bottom plate, which is optically transparent, to visible light is greater than 30%.

6. Said integrated nucleic acid analysis system according to claim 5, wherein said transmittance is greater than 90%.

7. Said integrated nucleic acid analysis system according to claim 2, wherein said first reservoir is filled with a lysis buffer and magnet beads, said second reservoir is filled with a magnetic bead washing solution, and said third reservoir is filled with a nucleic acid elution buffer.

8. Said integrated nucleic acid analysis system according to claim 1, wherein said gap of said at least one DMF device is filled with a filler fluid.

9. Said integrated nucleic acid analysis system according to claim 1, wherein said at least one DMF device is preloaded with a dried reagent.

10. Said integrated nucleic acid analysis system according to claim 1, wherein the instrument comprises:
a) at least one DMF device loading and unloading module;
b) at least one voltage control module, connected with said at least one DMF device to provide electric signals to said at least one DMF device for operating liquids or droplets in said at least one DMF device;
c) at least one magnet control module, located on an upper end or a lower end of said at least one DMF device, and configured to control one or more magnets, wherein each of said one or more magnets has at least two independent movement directions;
d) at least one temperature control module, configured to allow regions of said at least one DMF device to reach a specified temperature;
e) at least one light source, configured to perform optical excitation of nucleic acids in said at least one DMF device; and
f) at least one optical detection module, configured to perform optical measurement on at least one location of said at least one DMF device.

11. Said integrated nucleic acid analysis system according to claim 10, wherein the system further comprises a touch screen display and/or a central processing unit, wherein said touch screen display and said central processing unit are both electrically connected to said at least one optical detection module.

12. Said integrated nucleic acid analysis system according to claim 10, wherein each of said one or more magnets is a focusing magnet.

13. A method for measuring at least one target nucleic acid in a sample, comprising:
adding said sample to said at least one DMF device of said integrated nucleic acid analysis system according to claim 1;
performing assay operations on said sample to measure said at least one target nucleic acid, wherein said assay operations comprise the following:
1) mixing said sample with a lysis buffer;
2) adding magnetic beads to said sample;
3) mixing said magnetic beads;
4) washing said magnetic beads;
5) eluting said at least one target nucleic acid from said magnetic beads;
6) performing a thermal amplification on said at least one target nucleic acid; and
7) measuring fluorescence intensities of said at least one target nucleic acid.

14. Said method according to claim 13, wherein said washing of said magnetic beads comprises:
a) using one or more magnets in said instrument to transport said magnetic beads from said first reservoir, through a filler fluid, into a second reservoir, and to make said magnetic beads within said second reservoir to move according to a first specific trajectory;
b) making said magnetic beads to move, through a filler fluid, into a fourth reservoir, and making said magnetic beads within said fourth reservoir to move according to a second specific trajectory; and
c) making said magnetic beads to move, through said filler fluid, into said third reservoir.

15. (canceled)

16. Said method according to claim 13, wherein said washing of said magnetic beads comprises using one or more magnets in said system to transport said magnetic beads from said first reservoir, through a filler liquid, to said third reservoir directly.

17. Said method according to claim 13, wherein said eluting of said at least one target nucleic acid from said magnetic beads comprises:
a) moving one or more magnets away from said at least one DMF device to a specified distance, so that said magnetic beads is scattered in an elution solution;
b) moving said one or more magnets along a direction parallel to said at least one DMF device according to a specified trajectory within said third reservoir, to assist in washing said at least one target nucleic acid off said magnetic beads;
c) moving said one or more magnets to close to said at least one DMF device at said third reservoir;
d) gathering said magnetic beads in said third reservoir by moving one or more magnets according to said specified trajectory within said third reservoir;
e) moving said one or more magnets to close to said at least one DMF device at a location for discarding said magnetic beads; and
f) moving said one or more magnets away from said at least one DMF device.

18. Said method according to claim 13, wherein said performing of said thermal amplification on said at least one target nucleic acid; and said measuring of fluorescence intensities of said at least one target nucleic acid comprise:
a) dispensing one or more droplets from said third reservoir by utilizing said first set of electrowetting electrodes; and
b) moving said one or more droplets along pathways formed by a second set of electrowetting electrodes to perform said nucleic acid amplification and optical measurement.

19. Said method according to claim 13, wherein said measuring of fluorescence intensities of said at least one target nucleic acid is performed by said instrument automatically.

20. Said method according to claim 13, wherein said thermal amplification is a PCR amplification and/or an isothermal amplification.

21. (canceled)

22. Said method according to claim 18, wherein said optical measurement is performed after mixing eluted droplets with specified reagents.
